(19) United States
(12) Patent Application Publication
(10) Pub. No.: US 2021/0189458 A1
(43) Pub. Date: Jun. 24, 2021

(54) METHODS AND COMPOSITIONS FOR THE PURIFICATION OF UNBIASED RNA

(71) Applicant: ZYMO RESEARCH CORPORATION, Irvine, CA (US)

(72) Inventor: Stanislav FORMAN, Irvine, CA (US)

(21) Appl. No.: 16/757,297

(22) PCT Filed: Oct. 19, 2018
(86) PCT No.: PCT/US18/56735

§ 371 Date: Apr. 17, 2020

Related U.S. Application Data
(60) Provisional application No. 62/574,606, filed on Oct. 19, 2017.

(51) Int. Cl. C12Q 1/6806; C12N 15/10; C12Q 1/686

(52) U.S. Cl. CPC C12Q 1/6806; C12Q 1/686; C12N 15/1003

(57) ABSTRACT

Methods for non-biased KNA purification from biological samples are provided. In particular, this invention relates to methods of miRNA extraction and purification from blood, and its subsequent analysis.

This application claims benefit of priority to U.S. Provisional Application Ser. No. 62/574,606, filed Oct. 19, 2017, the entire contents of which are hereby incorporated by reference.

### BACKGROUND OF THE INVENTION

### 1. Field of the Invention

The present invention generally relates to biochemistry and molecular biology. More specifically, the invention relates to methods and compositions for purification of nucleic acid molecules.

### 2. Description of Related Art

A variety of protocols have been developed for purification of nucleic acids. One crucial step in many purification protocols involves the separation of nucleic acids from protein and lipid molecules that constitute cells and tissue matrices. Moreover for isolation of RNA in particular, the regents that maintain RNA stability during the purification process or preferably employed. However, previously purification processes from biological samples resulted in purified preparation that would selectively enriched for RNA of a particular size or type. Thus, there is a need in the art for methods for unbiased RNA purification from tissue samples, such as blood.

### SUMMARY OF THE INVENTION

In certain embodiments, the present disclosure provides methods for purifying unbiased RNA from blood sample comprising obtaining a biological sample, dissolved in a lysis reagent; lysing the sample dissolved in the lysis reagent; and purifying RNA from the mixture, wherein the purifying does not involve a RNA precipitation step.

In additional aspects, the method further comprises selectively analyzing micro RNAs from the purified RNA, wherein the purified RNAs provide a representative population of the RNA content of the original sample.

In some aspects, the biological sample is a liquid sample, a tissue sample, or a blood sample. In particular aspects, the blood sample is whole blood, plasma, serum, or buffy coat. In some aspects, obtaining the blood sample comprises collecting blood in a tube comprising the lysis reagent.

In certain aspects, the lysis reagent inactivates one or more microbes and nucleases in the blood sample. In some aspects, the one or more microbes comprise a virus, bacteria, and/or yeast. In certain aspects, the virus is influenza, ebola, and/or HSV. In some aspects, the bacteria is E. coli, B. subtilis, L. fermentum, E. faecalis, L. monocytogenes, P. aeruginosa, S. enterica, or S. aureus. In certain aspects, the yeast is C. neoformans and/or S. cerevisiae.

In some aspects, lysing and purifying are performed at 20-30° C., such as between about 21° C., 22° C., 23° C., 24° C., 25° C., 26° C., 27° C., 28° C., 29° C. and 30° C. In certain aspects, the lysing involves an incubation of period of at least 1 minute, such as for about 10 minutes to 2 hour, particularly about 5 minutes to 1 hour (e.g., 15 minutes, 20 minutes, 25 minutes, 30 minutes, 40 minutes, or 50 minutes). In some aspects, the incubation step comprises storing the sample at less than 10 degrees C. (e.g., 9° C., 8° C., 7° C., 6° C., 5° C., or 4° C.), for at least one day, such as for 24-72 hours, such as 2 days, 3 days, 4 days, or 5 days. In still further aspects, the incubation step can involve storage of the sample at less than 10 degrees for at least a one week, two weeks, a month two month, six months or a year.

In certain aspects, the lysis agent and the sample are mixed at 1:1 vol:vol ratio. In some aspects, the lysis agent and sample are mixed at a vol of 0.7-1.5 of lysis agent to vol of 0.7-1.5 of sample, such as 0.7:1, 0.8:1, 0.9:1, 1:0.7, 1:0.8, 1:0.9, 1:1.1, 1:1.2, 1:1.3, 1:1.4, 1:1.5, 1.1:1, 1.2:1, 1.3:1, 1.4:1, or 1.5:1 vol:vol of lysis agent to sample.

In specific aspects, the lysis agent comprises a chaotropic salt. In one specific aspect, the chaotropic salt is guanidinium thiocyanate. In additional aspects, the lysing step further comprises proteinase K digestion.

In some aspects, the lysing step further comprises agitation of the sample with one or more bead. In particular aspects, the one or more bead is a plurality of beads. In certain aspects, the plurality of beads are comprised of beads of different materials, sizes, or different shapes or the combination thereof. In some aspects, the beads are substantially spherical and comprise an average diameter of between 0.01 and 1.0 mm, such as 0.02, 0.03, 0.04, 0.05, 0.06, 0.07, 0.08, or 0.09 mm. In specific aspects, the beads of different sizes comprise beads that are between 0.25 and 0.75 mm (e.g., 0.3, 0.4, 0.5, 0.6 or 0.7 mm) and beads that are between 0.05 and 0.25 mm (e.g., 0.06, 0.07, 0.08, 0.09, 0.1, or 0.2 mm) in diameter. In particular aspects, the bead is substantially spherical. In some aspects, the bead is composed of a substantially non-reactive material. In one specific aspect, the bead is composed of a ceramic.

In certain aspects, the purifying step comprises applying the mixture to a silica spin column to bind the RNA to said column. In some aspects, the mixture is diluted in an equal volume of isopropanol prior to applying said sample to the column.

In additional aspects, purifying further comprises performing DNase I digestion. In some aspects, purifying further comprises removal of the chaotropic salt. In certain aspects, purifying further comprises washing the column with a buffer comprising ethanol or isopropanol. In particular aspects, purifying does not comprise alcohol precipitation of the RNA or phase separation. In some aspects, purifying comprises eluting the RNA into RNase-free water. In certain aspects, the purified RNA is essentially free of DNA. In some aspects, the purified RNA comprises micro RNA, small interfering RNA, and/or piwi RNA. In certain aspects, the purified RNA comprises RNA molecules less than 200 nucleotides in length.

In some aspects, analyzing micro RNAs comprises performing microarray analysis, single cell assays, northern blotting, or qRT-PCR. In particular aspects, analyzing micro RNAs comprises constructing a library for miRNA sequencing and performing next generation miRNA sequencing on said library. In some aspects, constructing a library comprises ligating adaptors to each end of the micro RNAs. In specific aspects, the adaptors comprise barcodes. In certain aspects, the method further comprises performing Nanostring nCounter analysis on the sequencing results.

In certain aspects, the method further comprises performing unbiased miRNA functional enrichment analysis. In some aspects, the analysis comprises using a target prediction program, gene annotation data, and applying statistical analysis.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Graphs show quantitative detection of miR126 by direct hybridization (n-Counter; Nanostring) or RNA-seq (Illumina), comparing samples collected in a commercial storage buffer (Paxgene, PreAnalytiX) followed by a precipitating RNA isolation method (RNeasy; Qiagen) (left), a commercial storage buffer (Paxgene) followed by a non-precipitating RNA isolation method (Trizol) (middle), and storage in DNA/RNA shield and isolation with the Zymo QuickRNA kit (Right).

FIG. 2: Graphs show quantitative detection of let7a-5p, miR-423-5P or miR145a by direct hybridization (n-Counter; Nanostring), comparing storage and isolation with Paxgene and RNeasy (left), Paxgene and Trizol (middle), and DNA/RNA shield and Zymo QuickRNA (right).

FIG. 3: Graphs show quantitative detection of has-mrR-92a-3p, has-miR-4732-3p, has-miR-19b-3p or has-miR-197-3p by miRNA sequencing (Illumina), comparing storage and isolation with Paxgene and RNeasy (left), Paxgene and Trizol (middle), and DNA/RNA shield and Zymo QuickRNA (right).

FIG. 4: Graphs and tables show quantitative detection of miR-191 or let7b-5p by direct hybridization (nCounter; Nanostring), comparing storage and isolation with Paxgene and RNeasy (left), Paxgene and Trizol (middle), and DNA/RNA shield and Zymo QuickRNA (right).

FIG. 5: Graphs and tables show quantitative detection of miR-191 or let7b-5p by miRNA sequencing (Illumina), comparing storage and isolation with Paxgene and RNeasy (left), Paxgene and Trizol (middle), and DNA/RNA shield and Zymo QuickRNA (right).

FIG. 6: Graphs show quantitative detection of let7 or miR-191 by miRNA specific RT-qPCR (Quantabio), comparing storage and isolation with Paxgene and RNeasy (left), Paxgene and Trizol (middle), and DNA/RNA shield and Zymo QuickRNA (right).

### DESCRIPTION OF ILLUSTRATIVE EMBODIMENTS

### I. The Present Embodiments

The instant application provide for the first time a method of purifying RNA from a sample that non-biased and able to capture a more represented proportion of small RNAs (such miRNAs) from a biological sample. N particular the methods detailed herein provide improved RNA purification methods that do not include a RNA precipitation step.

### II. Reagents and Kits

Kits may comprise suitably aliquoted reagents of the present invention, such as an acid-phenol denaturing solvent, one or more binding agent and a silica substrate. Additional components that may be included in a kit according to the invention include, but are not limited to, one or more wash buffer, an elution buffer, a proteinase composition, DNase and/or RNase inhibitors, DNase or RNase enzymes, oligonucleotide primers, reference samples (e.g., samples comprising known amounts of DNA or RNA), distilled water, DEPC-treated water, probes, sample vials, polymerase, and instructions for nucleic acid purification. In certain further aspects, additional reagents for DNA and/or RNA clean-up may be included.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing reagent containers in close confinement for commercial sale. Such containers may include cardboard containers or injection or blow-molded plastic containers into which the desired vials are retained.

When the components of the kit are provided in one or more liquid solutions, the liquid solution is an aqueous solution, with a sterile aqueous solution being preferred. However, the components of the kit may be provided as dried powder(s). When reagents and/or components are provided as a dry powder, the powder can be reconstituted by the addition of a suitable solvent. It is envisioned that the solvent may also be provided in another container means.

### III. Samples

The level of at least one miRNA gene product can be measured in cells of a biological sample. A biological sample may comprise a cell, milk, blood, serum, plasma, ascites, cyst fluid, pleural fluid, peritoneal fluid, cerebral spinal fluid, tears, urine, feces, saliva, sputum, virus, tissue, plants, or combinations thereof.

For example, a tissue sample can be removed from a subject by conventional biopsy techniques. In another example, a blood sample can be removed from a subject, and white blood cells can be isolated for RNA extraction by standard techniques. The blood or tissue sample is preferably obtained from the subject prior to initiation of radiotherapy, chemotherapy or other therapeutic treatment. A corresponding control tissue or blood sample can be obtained from unaffected tissues of the subject, from a normal human individual or population of normal individuals, or from cultured cells corresponding to the majority of cells in the subject's sample. The control tissue or blood sample may then be processed along with the sample from the subject, so that the levels of miR gene product produced from a given miR gene in cells from the subject's sample can be compared to the corresponding miR gene product levels from cells of the control sample.

In another example, plant tissues may be removed and RNA may be extracted by standard techniques. Plant tissues may be isolated from a variety of tissues in the same plant and miRNA levels compared within tissues of the same plant. In another example, miRNA may be isolated from the same tissue of both experimental and control plants form control plants for processing in parallel so that the levels of miRNA gene products produced from cells of specific tissues can be compared.

### IV. Downstream Processing

miRNA may be detected and analyzed in a variety of ways. Isolated miRNA may be analyzed by Northern blot to determine the presence or absence of a miRNA of interest, or determine the quantity of a miRNA of interest with relation to a control. Isolated miRNA may be hybridized to a solid support for detection. Isolated miRNA may be detected by qRT-PCR. miRNA may be hybridized to a probe and amplified to aid in detection. Amplified miRNA may be detected by qPCR, northern blot, or by sequencing. miRNA may be ligated to at least one oligonucleotide, reverse transcribed and amplified. Amplified miRNA products may be detected by qPCR. miRNA may be detected and quantified using by hybridization to a microarray. miRNA may be detected and analyzed by miRNA-seq. miRNA-seq requires the isolation of miRNA from total RNA, reverse transcription of the miRNA to cDNA, and sequencing of the cDNA product by Sanger sequencing, pyrosequencing, or next generation sequencing.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Detection of miR126

Sample collection. Human Blood samples were collected from Cardinal Biologicals and drawn directly into respective Blood Preservative Tubes (Paxgene, PreAnalytiX or DNA/RNA Shield, Zymo Research).

RNA purification. RNA was purified from the PAXgene preserved blood samples using either the PAXgene miRNA purification kit (#763134; Qiagen) (FIG. 1, col. 1), or using the Trizol extraction with BCP followed by RNA purification from the aqueous phase using the RNA Clean & Concentrator kit (#R1015; Zymo Research) (FIG. 1, col. 2). Blood collected into DNA/RNA Shield was purified using the Quick-RNA Whole Blood kit (#R1201; Zymo Research) (FIG. 1, col. 3).

Library preparation and sequencing. miRNA-seq libraries were prepared using RapidSeg™ High Yield Small RNA Sample Prep Kit (#KS074012; Biochain). Briefly, two sequential ligation reactions were performed to attach first 3′ adapter and then 5′ adapter to RNA molecules. This was followed by a reverse transcription reaction and amplification with Illumina index primers. Then 130-150 bp fragments were purified from a 2% 1:1 NuSieve:agarose gel (Zymoclean™ Gel DNA Recovery Kit, Zymo Research, cat. #D4001). The purified libraries were run on Agilent 2200 TapeStation to confirm that the library fragments were of desired size. The libraries were sequenced on the Illumina HiSeq platform. Approximately 2-5 million 50 bp single-end reads were obtained for each library.

Detection of miR126. The use of precipitation based preservation and purification methods did not yield a detectable signal of miR126 using either a direct hybridization method (nCounter; Nanostring) or the Illumina miRNA-seq pipeline (FIG. 1, col. 1). Direct purification can partially restore the nucleic acid (miRNA) recovery from the precipitation based preservation reagents (FIG. 1, col. 2). Direct purification from the non-precipitating preservation reagent (DNA/RNA shield) yields the best recovery of the miRNA from the blood samples (FIG. 1, col. 3).

### Example 2—Nanostring Analysis of Selected miRNAs

Sample collection. Human Blood samples were collected from Cardinal Biologicals and drawn directly into respective Blood Preservative Tubes (Paxgene, PreAnalytiX or DNA/RNA Shield, Zymo Research).

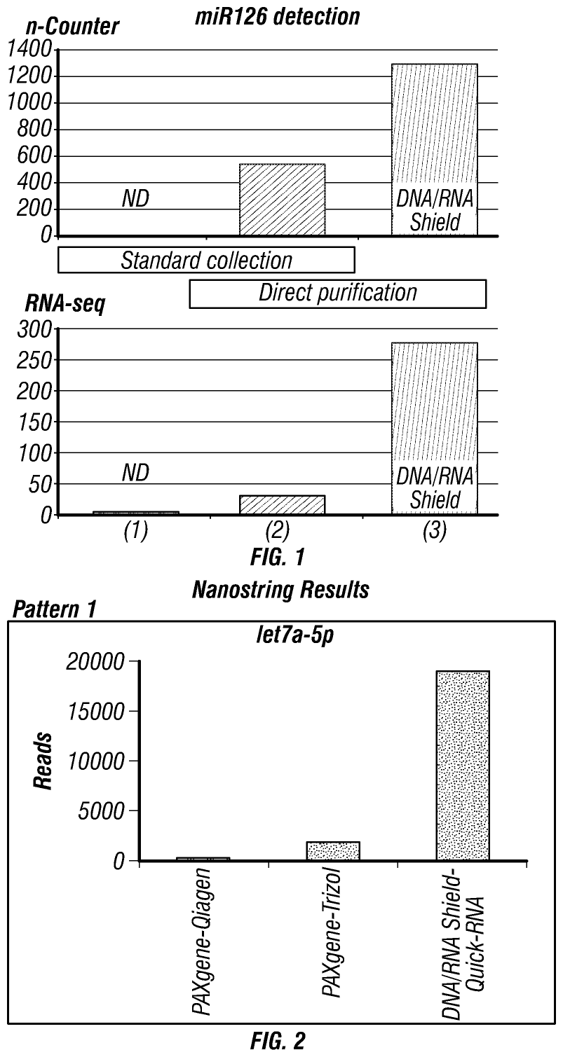

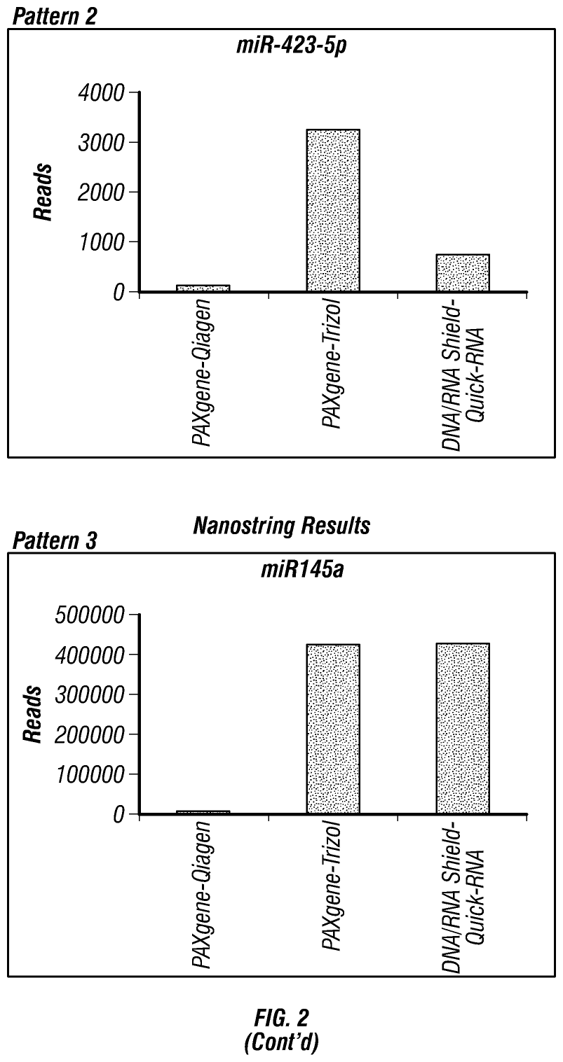

Nanostring analysis of selected miRNAs. RNA isolation and library preparation were performed as in Example 1 for each of the preservation methods. Direct hybridization data was analyzed using the Nanostring nCounterNorm package in R. miRNA with fewer than 100 reads were excluded from analysis, as lower reads showed to be random/inconsistent. 3 patterns were found and evaluated. Pattern 1 shows that non-precipitating preservation with DNA/RNA Shield, followed by direct purification of miRNA (DNA/RNA Shield—Quick-RNA), results in significantly more sequencing reads than precipitation based preservation methods with precipitation based purification (PAXgene-Qiagen) or with direct purification (PAXgene-Trizol). This pattern was evident when examining let7a-5p (FIG. 2). Pattern 2 finds the PAXgene-Trizol method results in more sequencing reads than PAXgene-Qiagen method or the DNA/RNA Shield—Quick RNA method. An example miRNA detected with this pattern is miR-423-5p (FIG. 2). Pattern 3 found the PAXgene-Trizol method and the DNA/RNA Shield—Quick-RNA method to generate similar numbers of reads for an identified miRNA, both with far more reads than the PAXgene-Qiagen method. miRNA145a is an example a miRNA with Pattern 3 (FIG. 2). Pattern 1 was seen for about 80% of targets, while Patterns 2 and 3 were seen for 11% and 9% of targets, respectively (Table 1). This distribution indicates that for the majority of samples, it is preferable to store the samples using a non-precipitating preservation method, and a direct purification method.

TABLE 1
Percent distribution of the patterns observed between
three storage and purification methods.

| Pattern 1 | pattern 2 | pattern 3 |
|---|---|---|
| 80% | 11% | 9% |

### Example 3—miRNA-Seq Analysis of Selected miRNAs

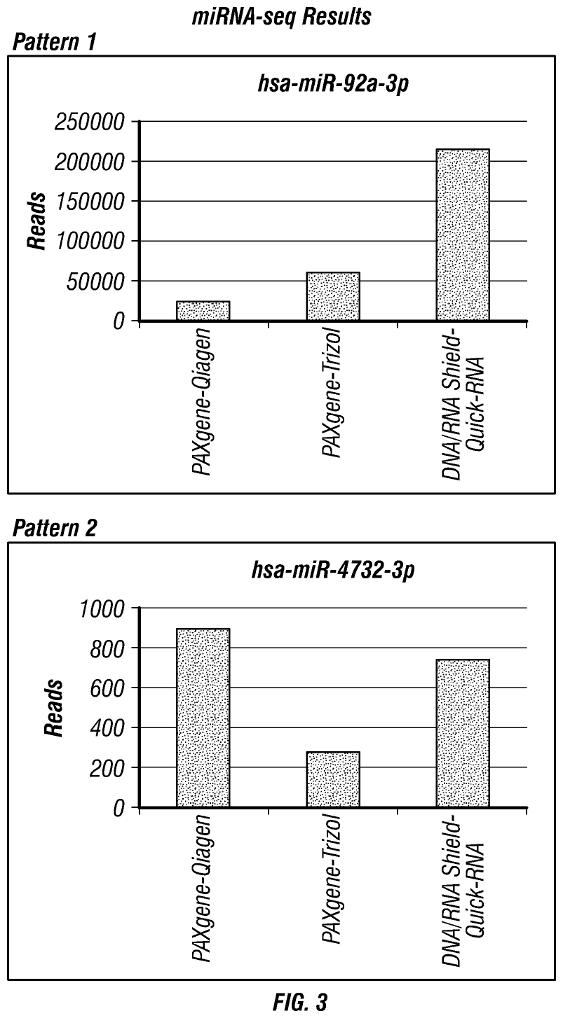

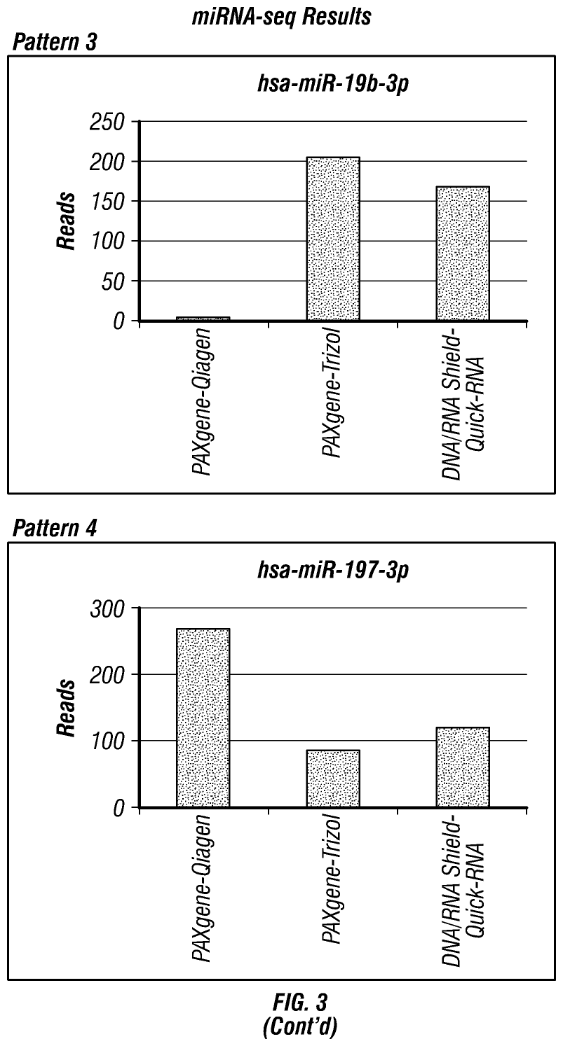

Sequencing an analysis of selected miRNAs. Sample collection and RNA isolation were performed as above. Sequencing was performed on an IlluminaHiSeq 1500 with 2-5 million reads per library. miRNA-seq data was analyzed. 4 distinct patterns were found. The most common is Pattern 1, showing significantly more reads generated for a given target RNA when prepared by the non-precipitation preservation and direct purification method (DNA/RNA Shield—QuickRNA). An example of Pattern 1 is has-miR-92a-3p (FIG. 3). Pattern 2 shows similar amounts of miRNA reads for the precipitating preservation followed by precipitating purification method (PAXgene-Qiagen) and the DNA/RNA Shield—QuickRNA method, and can be seen for has-miR-4732-3p (FIG. 3). Pattern 3 indicates similar amounts of reads for the precipitating preservation and direct purification method and the DNA/RNA Shield—QuickRNA method, and is found for has-miR-19b-3p (FIG. 3). The least common pattern was pattern 4, finding the PAXgene-Qiagen method to generate the most reads for a given target, such as has-miR-197-3p (FIG. 3). Pattern 1 accounted for 89% of targets, with Pattern 2 accounting for 7%, Pattern 3 for 3%, and Pattern 4 for 1%, indicating that for the majority of targets, using a non-precipitating preservation method followed by a direct purification method is superior to the others (Table 2).

% Distribution of the Patterns observed between
three purification methods.

| Pattern 1 | pattern 2 | pattern 3 | Pattern 4 |
|---|---|---|---|
| 89% | 7% | 3% | 1% |

### Example 4—Interexperimental Marker Consistency

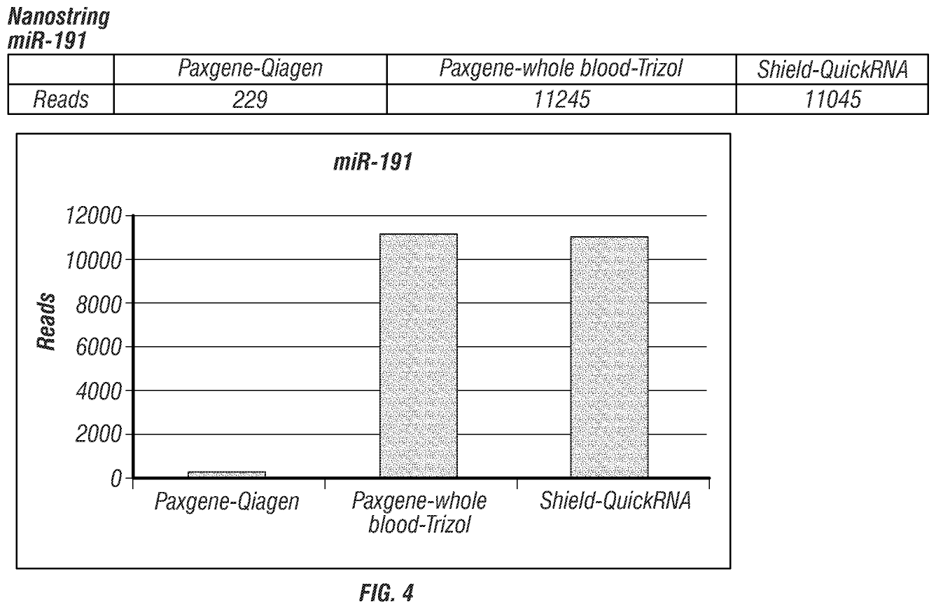

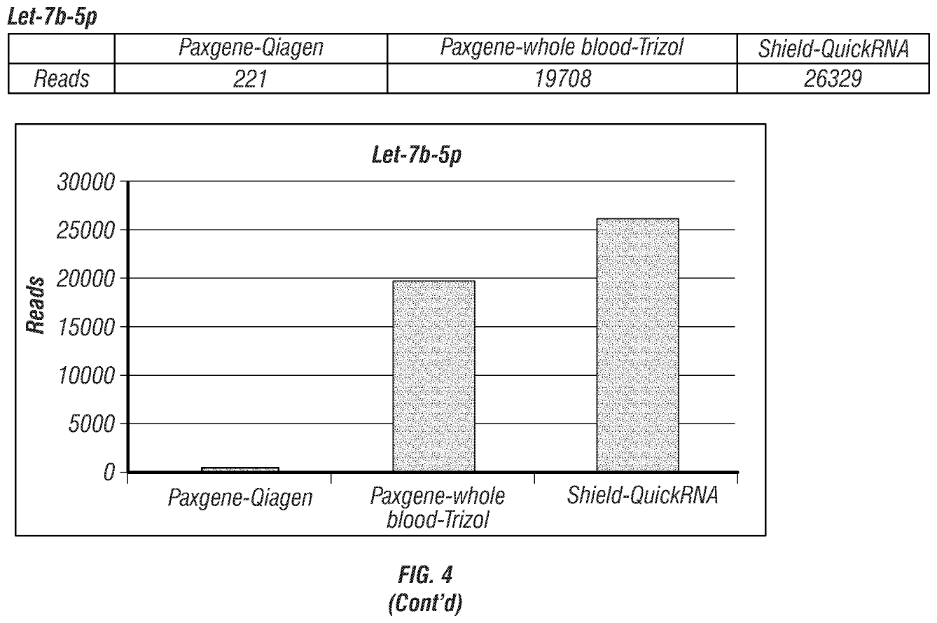

Nanostring analysis. Sample collection, RNA isolation, and sequencing were performed as above. RNA-seq data was analyzed using the nCounter analysis by Nanostring to interrogate miR-191 and Let-7b-5p, generating FIG. 4. Briefly, non-precipitating preservation followed by direct isolation (Shield—QuickRNA) resulted in similar or greater numbers of sequencing reads than precipitating preservation followed by direct isolation (PAXgene-whole blood-Trizol) (FIG. 4, miR-191 and Let-7b-5p), and significantly greater numbers of reads than precipitating preservation followed by precipitating RNA isolation (PAXgene-Qiagen) (FIG. 4, both).

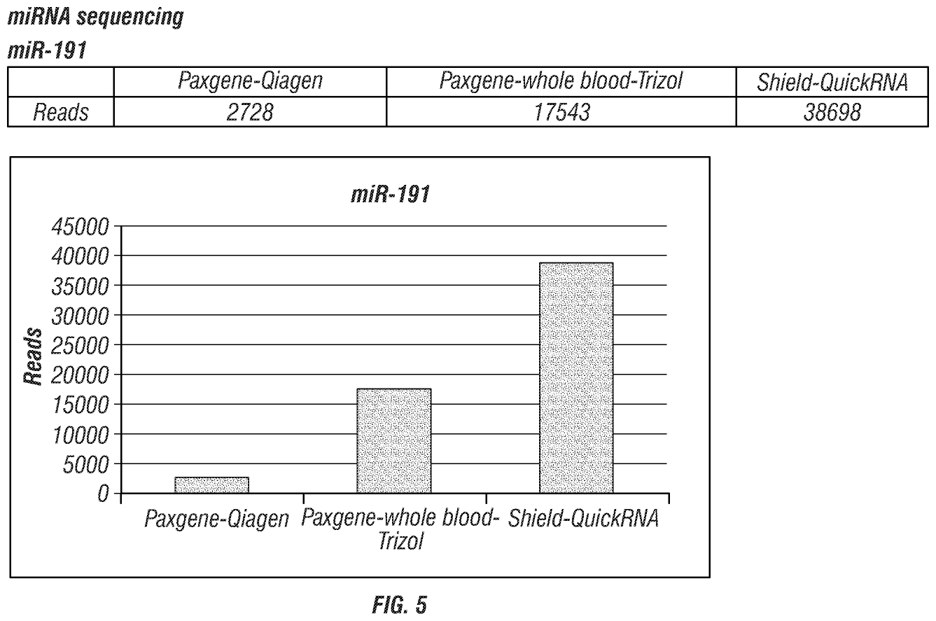

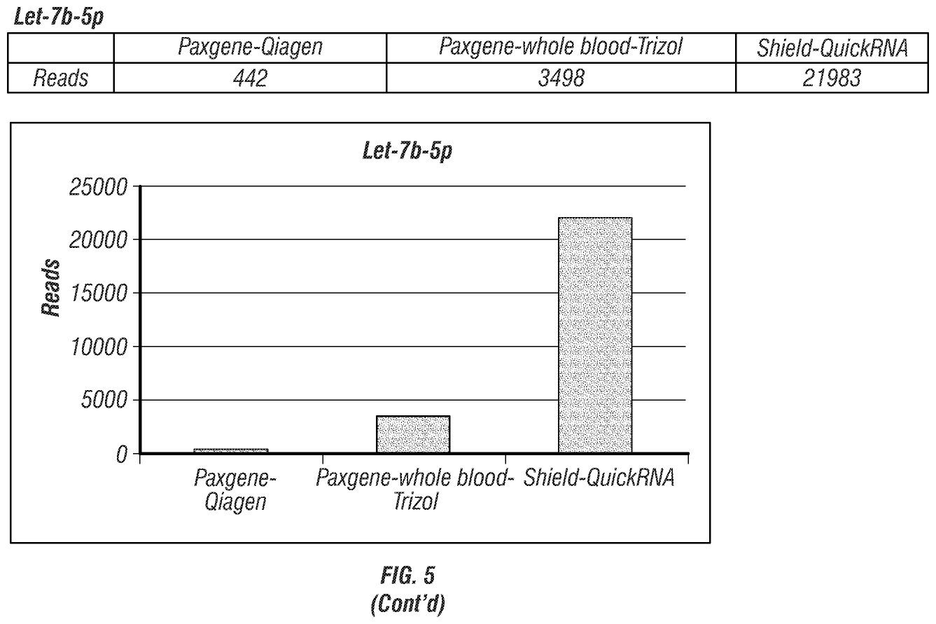

miRNA-seq analysis of miR-191 and Let-7b-5p. Sample collection, RNA isolation, and sequencing were performed as above. RNA-seq data was analyzed to interrogate miR-191 and Let-7b-5p, generating FIG. 5. Briefly, non-precipitating preservation followed by direct isolation (Shield—QuickRNA) resulted in greater numbers of sequencing reads than the precipitating preservation method followed by either direct isolation (PAXgene-whole blood-Trizol) or precipitating RNA isolation (PAXgene-Qiagen) (FIG. 5, both).

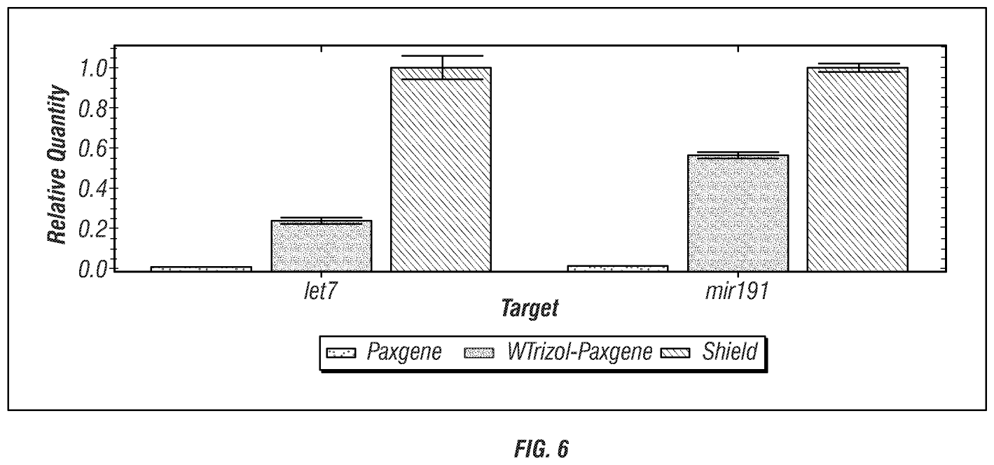

miRNA RT-qPCR of Let-7b-5p and miR-191. RT-qPCR was performed using the qScript microRNA cDNA Synthesis Kit (#95107-025; Quantabio) (FIG. 6). There was increased miRNA (let7-b-5p and miR-191) recovery and detection from samples stored in DNA/RNA Shield and purified using the Zymo Research Quick-RNA whole blood Miniprep kit, consistent with the results from Nanostring (FIG. 4) and miRNA-seq (FIG. 5) analyses.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

What is claimed is:

1. A method for purifying unbiased RNA from blood sample comprising:
(a) obtaining a biological sample, dissolved in a lysis reagent;
(b) lysing the sample dissolved in the lysis reagent; and
(c) purifying RNA from the mixture, wherein the purifying does not involve a RNA precipitation step; and
(d) selectively analyzing micro RNAs from the purified RNA, wherein the purified RNAs provide a representative population of the RNA content of the original sample.

2. The method of claim 1, wherein the blood sample is whole blood, plasma, serum, or buffy coat.

3. The method of claim 1, wherein obtaining the blood sample comprises collecting blood in a tube comprising the lysis reagent.

4. The method of any one of claims 1-3, wherein the lysis reagent inactivates one or more microbes and nucleases in the blood sample.

5. The method of claim 4, wherein the one or more microbes comprise a virus, bacteria, and/or yeast.

6. The method of claim 4, wherein the virus is influenza, ebola, and/or HSV.

7. The method of claim 5, wherein the bacteria is E. coli, B. subtilis, L. fermentum, E. faecalis, L. monocytogenes, P. aeruginosa, S. enterica, or S. aureus.

8. The method of claim 5, wherein the yeast is C. neoformans and/or S. cerevisiae.

9. The method of claim 1, wherein lysing and purifying are performed at 20-30° C.

10. The method of claim 1, step (b) involves an incubation of period or at least 1 minute.

11. The method of claim 10, wherein the incubation is for 5 minutes to 1 hour.

12. The method of claim 10, wherein the incubation is for 10 minutes to 2 hours.

13. The method of claim 10, wherein the incubation step comprises storing the sample at less than 10 degrees C. for at least one day.

14. The method of claim 1, wherein the lysis agent and the sample are mixed at a volume of 0.7-1.5 of lysis agent to a volume of 0.7-1.5 of sample.

15. The method of claim 1, wherein the lysis agent and the sample are mixed at a 0.7:1, 0.8:1, 0.9:1, 1:0.7, 1:0.8, 1:0.9, 1:1.1, 1:1.2, 1:1.3, 1:1.4, 1:1.5, 1.1:1, 1.2:1, 1.3:1, 1.4:1, or 1.5:1 vol:vol of lysis agent to sample.

16. The method of claim 15, wherein the lysis agent and the sample are mixed at 1:1 vol:vol ratio.

17. The method of claim 1, wherein the lysis agent comprises a chaotropic salt.

18. The method of claim 17, wherein the chaotropic salt is guanidinium thiocyanate.

19. The method of claim 1, wherein the lysis agent contains phenol.

20. The method of claim 1, wherein the lysis agent is essentially free of phenol.

21. The method of claim 20, wherein the lysis agent is free of phenol.

22. The method of claim 1, wherein the lysing step further comprises proteinase K digestion.

23. The method of claim 1, wherein the lysing step further comprises agitation of the sample with one or more bead.

24. The method of claim 23, wherein the one or more bead is a plurality of beads.

25. The method of claim 24, wherein the plurality of beads comprised of beads of different materials, sizes, or different shapes or the combination thereof.

26. The method of claim 24, wherein the beads are substantially spherical and comprise an average diameter of between 0.01 and 1.0 mm.

27. The method of claim 24, wherein the beads of different sizes comprise beads that are between 0.25 and 0.75 mm and beads that are between 0.05 and 0.25 mm in diameter.

28. The method of claim 24, wherein the bead is substantially spherical.

29. The method of claim 24, wherein the bead is composed of a substantially non-reactive material.

30. The method of claim 24, wherein the bead is composed of a ceramic.

31. The method of claim 1, wherein the purifying step comprises applying the mixture to a silica spin column to bind the RNA to said column.

32. The method of claim 31, wherein the mixture is diluted in an equal volume of isopropanol prior to applying said sample to the column.

33. The method of any one of claims 9-32, wherein purifying further comprises performing DNase I digestion.

34. The method of claim 33, wherein purifying further comprises removal of the chaotropic salt.

35. The method of claim 33, wherein purifying further comprises washing the column with a buffer comprising ethanol or isopropanol.

36. The method of any one of claims 1-35, wherein purifying does not comprise alcohol precipitation of the RNA or phase separation.

37. The method of claim 36, wherein purifying comprises eluting the RNA into RNase-free water.

38. The method of claim 37, wherein the purified RNA is essentially free of DNA.

39. The method of claim 37, wherein the purified RNA comprises micro RNA, small interfering RNA, and/or piwi RNA.

40. The method of claim 37, wherein the purified RNA comprises RNA molecules less than 200 nucleotides in length.

41. The method of claim 1, wherein analyzing micro RNAs comprises performing microarray analysis, single cell assays, northern blotting, or qRT-PCR.

42. The method of claim 1, wherein analyzing micro RNAs comprises constructing a library for miRNA sequencing and performing next generation miRNA sequencing on said library.

43. The method of claim 42, wherein constructing a library comprises ligating adaptors to each end of the micro RNAs.

44. The method of claim 43, wherein the adaptors comprise barcodes.

45. The method of claim 42, further comprising performing Nanostring nCounter analysis.

46. The method of claim 42, further comprising performing unbiased miRNA functional enrichment analysis.

47. The method of claim 46, wherein said analysis comprises using a target prediction program, gene annotation data, and applying statistical analysis.
